SEQUENCE LISTING (70 sequences)

SEQ ID NO: 1 - protein, 31 aa, synthetic construct
HAEGTFTSDVSSYLEGQAAKEFIAWLVKGRG

SEQ ID NO: 2 - protein, 31 aa, synthetic construct
HGEGTFTSDVSSYLEEQAAKEFIAWLVKGGG

SEQ ID NO: 3 - protein, 40 aa, synthetic construct
  MOD_RES 2: note = Aib
  MOD_RES 13: note = Aib
  MOD_RES 21: note = diacid-gamma-Glu-(AEEA)2
YXEGTFTSDYSIXLDKIAQKXAFVQWLIAGGPSSGAPPPS

SEQ ID NO: 4 - protein, 39 aa, synthetic construct
YGEGTFTSDYSIYLDKIAQKAFVQWLIAGGPSSGAPPPS

SEQ ID NO: 5 - protein, 39 aa, synthetic construct
YGEGTFTSDYSIQLDKIAQKAFVQWLIAGGPSSGAPPPS

SEQ ID NO: 6 - protein, 350 aa, synthetic construct
QVQLVETGGGLVQAGGSLRLSCAASGRTFSSYRMGWFRQAPGKEREFVAVISRSGDVTTYAHSVKGRFTVSRDNAKNTVYLQMNSLKPEDTAVYYCAAGLAANTANYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 352 aa, synthetic construct
EVQLQESGGGLVQAGGSLRLSCAASGSISSLNGIWYRQAPGKQREVVAGITSGGSTNYADSVKGRFTISRDNAKNTVYLQMNGLKPEDTAIYYCNTVRNWDIRTALSRSGGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 352 aa, synthetic construct
QVQLVESGGGLVQPGGSLRLSCAASGSIFAINVMGWYRQASGNQRELVAGITTGGARDYADSVKGRFTISRDNAKNTVYLSMNSLKPEDTAVYYCNLHAPKYIDSRYDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 350 aa, synthetic construct
EVQVQESGGGLVQAGGSLRLSCAASGRSFRSDRMGWFRQAPGKEREFVAVISRGGGVTTYADSVKGRFTVSRDDAKNTVDLQMNSLKPEDTAVYYCAAGITTKAANYWGMGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 347 aa, synthetic construct
QVQLVESGGGLVQPGGSLRLSCAASGSVFSRNFMGWYRQAPGKQRELVAYITVDGGTRYADSVKGRFTISRDNAKNTVYLQMNSLKPEDTAVYYCNRAGTTPYAWGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 353 aa, synthetic construct
QVRLVESGGGLVQAGGSLRLSCTASGRTFSSYSVGWFRQAPGKERAFVAVISKSGGITTYADSVKGRFIISRDNAKNTVYLQMNSLKPEDTAVYYCAASGSSLRLADSGSWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 350 aa, synthetic construct
EVQLVESGGGLVQAGGSLRLSCVASGRTFGNYRMAWFRQAPGKEREFVAVISRNGGITTYADSAKGRFTLSRDNAKNAVYLQMNSLKPEDTAVYYCTGGRNLGTWDYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 350 aa, synthetic construct
QVQLVESGGGLVQAGGSLRLSCVASGRTFSSGRMAWFRQAPGKEREFVAVISQNGGVTTYAASVKGRFTVSRDNAKNTVYLQMNSLKPEDTAVYYCAAGVTTNTANYWGQGTRVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 356 aa, synthetic construct
EVQLQESGGGLVQAGGSLRLSCVVSGRTFGSLRMAWFRQAPGKEREFVSIISKSGGITTYADSVKGRFTISRDNAKNTVYLQMNSLKPEDTAVYYCAADASIRPRTNPSDYDGWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 350 aa, synthetic construct
QVQLVESGGGLVQPGGSLRLSCVASGNIFSSSIMGWYRQAPGKQREAVAFITNGGSTDYADSVKGRFTISRDNAKNTTYLQMNSLKFEDTAVYYCNLRNQRNFQEGSLGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 345 aa, synthetic construct
EVQLVESGGGLVQAGGSLRLSCAASGIDFRADVMAWYRQAPGKQREVVAAITAYGDTDYVDSVKGRFTISRDNAKNTVYLLMNSLKPEDTAVYYCNVPVRRYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 350 aa, synthetic construct
EVQVVESGGGLVQPGGSLRLSCAASGSYFSINVMAWYRQAPGKQREVVAAISRGGFADYAQFAKGRFTISGDNAKNTVSLQMNSLKPEDTAVYYCNARRAWDTAINYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 354 aa, synthetic construct
QVQLVESGGGLVQPGGSLRLSCAASGSIHSINILGWYRQAPGDHRELVAGITTGGSTDYADSVKDRFTISRDNAKNTVYLQMNDLKPADTAVYYCNLRQSSTRLTWRPYDSWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 354 aa, synthetic construct
QVQLVESGGGLVQPGGSLRLSCAASGFTFSSTSMTWVRQRPGRGLEWVSSIFSDGRSTYYADSLKGRVTISRDNAENTLYLQMNSLQSEDTAVYYCAKGLPSLRVGVMGLLRGQGTLVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 345 aa, synthetic construct
EVRLVESGGGLVQAGGSLRLSCAASGISFRADVMAWYRQAPGKQREVVAAITSYGDADYADSVKGRFTISRDNAKNTVYLQMNSLKPEDTAVYYCNVPARRYWGQGTQVTVSSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 115 aa, synthetic construct
QVQLVESGGGLVQPGGSLRLSCAASGSVFSRNFMGWYRQAPGKQRELVAYITVDGGTRYADSVKGRFTISRDNAKNTVYLQMNSLKPEDTAVYYCNRAGTTPYAWGQGTLVTVSS

SEQ ID NO: 22 - protein, 409 aa, synthetic construct
QVQLVESGGGLVQPGGSLRLSCAASGSVFSRNFMGWYRQAPGKQRELVAYITVDGGTRYADSVKGRFTISRDNAKNTVYLQMNSLKPEDTAVYYCNRAGTTPYAWGQGTLVTVSSGGGGSGGGGSGGGGSRGERHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 23 - protein, 408 aa, synthetic construct
QVQLVESGGGLVQPGGSLRLSCAASGSVFSRNFMGWYRQAPGKQRELVAYITVDGGTRYADSVKGRFTISRDNAKNTVYLQMNSLKPEDTAVYYCNRAGTTPYAWGQGTLVTVSSGGGGSGGGGSGGGGSRKRHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 24 - protein, 119 aa, synthetic construct
QVQLQQPGAELVKPGASVKLSCRASGYTFTSNWMHWVKQRPRQGLEWIGEINPSNGRSNYNEKFKTKATLTVDKSSSTAYMQLSSLTSEDSAVYYCARFYYGTSWFAYWGQGTLVAVSA

SEQ ID NO: 25 - protein, 113 aa, synthetic construct
DVVMTQTPLSLPVSLGDQASISCRSSQSLVHSNGDTYLHWYLQKPGQSPKLLIYKVSNRFSGVPDRFSGSGSGTDFTLKISRVEAADLGVYFCSQSTHVPPFTFGGGTKLEIK

SEQ ID NO: 26 - protein, 400 aa, synthetic construct
QVQLVESGGGLVQPGGSLRLSCAASGSVFSRNFMGWYRQAPGKQRELVAYITVDGGTRYADSVKGRFTISRDNAKNTVYLQMNSLKPEDTAVYYCNRAGTTPYAWGQGTLVTVSSGGGGSGGGGSGGGGSRGERHGEGTFTSDVSSYLEEQAAKEFIAWLVKGGGGGGGSGGGGSGGGGSDVVMTQTPLSLPVSLGDQASISCRSSQSLVHSNGDTYLHWYLQKPGQSPKLLIYKVSNRFSGVPDRFSGSGSGTDFTLKISRVEAADLGVYFCSQSTHVPPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 445 aa, synthetic construct
QVQLQQPGAELVKPGASVKLSCRASGYTFTSNWMHWVKQRPRQGLEWIGEINPSNGRSNYNEKFKTKATLTVDKSSSTAYMQLSSLTSEDSAVYYCARFYYGTSWFAYWGQGTLVAVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 399 aa, synthetic construct
QVQLVESGGGLVQPGGSLRLSCAASGSVFSRNFMGWYRQAPGKQRELVAYITVDGGTRYADSVKGRFTISRDNAKNTVYLQMNSLKPEDTAVYYCNRAGTTPYAWGQGTLVTVSSGGGGSGGGGSGGGGSRKRHGEGTFTSDVSSYLEEQAAKEFIAWLVKGGGGGGGSGGGGSGGGGSDVVMTQTPLSLPVSLGDQASISCRSSQSLVHSNGDTYLHWYLQKPGQSPKLLIYKVSNRFSGVPDRFSGSGSGTDFTLKISRVEAADLGVYFCSQSTHVPPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 266 aa, synthetic construct
HGEGTFTSDVSSYLEEQAAKEFIAWLVKGGGGGGGSGGGGSGGGGSDVVMTQTPLSLPVSLGDQASISCRSSQSLVHSNGDTYLHWYLQKPGQSPKLLIYKVSNRFSGVPDRFSGSGSGTDFTLKISRVEAADLGVYFCSQSTHVPPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 118 aa, synthetic construct
DVQLVESGGGLVQPGGSRKLSCAASGFTFSSFGMHWVRQAPEKGLEWVAYISSGSSTIYYADTVKGRFTISRDNPKNTLFLQMTSLRSEDTAMYYCARGKYPTWIAYWGQGTLVTVSA

SEQ ID NO: 31 - protein, 113 aa, synthetic construct
DIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELK

SEQ ID NO: 32 - protein, 120 aa, synthetic construct
QVTLKESGPGILQPSQTLSLTCSFSGFSLSTSGTGVGWIRQPSGKGLEWLSHIWWDDVKRYNPALKSRLTISRDTSYSQVFLRIASVDTADTATYYCARILDGTGPMDYWGQGTSVTVSS

SEQ ID NO: 33 - protein, 108 aa, synthetic construct
QIVLTQSPAIMSASPGEKVTMTCSASSRVTYMHWYQQRSGTSPKRWIYDTSKLASGVPARFSGSGSGTSYSLTISSMEAEDAATYYCQQWGNNPQYTFGGGTRLEIKR

SEQ ID NO: 34 - protein, 122 aa, synthetic construct
QVQLVESGGGLVQPGGSLRLSCAASGSIHSINILGWYRQAPGDHRELVAGITTGGSTDYADSVKDRFTISRDNAKNTVYLQMNDLKPADTAVYYCNLRQSSTRLTWRPYDSWGQGTQVTVSS

SEQ ID NO: 35 - protein, 415 aa, synthetic construct
QVQLVESGGGLVQPGGSLRLSCAASGSIHSINILGWYRQAPGDHRELVAGITTGGSTDYADSVKDRFTISRDNAKNTVYLQMNDLKPADTAVYYCNLRQSSTRLTWRPYDSWGQGTQVTVSSGGGGSGGGGSGGGGSRGERYGEGTFTSDYSIYLDKIAQKAFVQWLIAGGPSSGAPPPSGGGGSGGGGSGGGGSDIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 444 aa, synthetic construct
DVQLVESGGGLVQPGGSRKLSCAASGFTFSSFGMHWVRQAPEKGLEWVAYISSGSSTIYYADTVKGRFTISRDNPKNTLFLQMTSLRSEDTAMYYCARGKYPTWIAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 37 - protein, 414 aa, synthetic construct
QVQLVESGGGLVQPGGSLRLSCAASGSIHSINILGWYRQAPGDHRELVAGITTGGSTDYADSVKDRFTISRDNAKNTVYLQMNDLKPADTAVYYCNLRQSSTRLTWRPYDSWGQGTQVTVSSGGGGSGGGGSGGGGSRKRYGEGTFTSDYSIYLDKIAQKAFVQWLIAGGPSSGAPPPSGGGGSGGGGSGGGGSDIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 274 aa, synthetic construct
YGEGTFTSDYSIYLDKIAQKAFVQWLIAGGPSSGAPPPSGGGGSGGGGSGGGGSDIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 444 aa, synthetic construct; identical to SEQ ID NO: 36

SEQ ID NO: 40 - protein, 274 aa, synthetic construct; identical to SEQ ID NO: 38

SEQ ID NO: 41 - protein, 268 aa, synthetic construct
YGEGTFTSDYSIYLDKIAQKAFVQWLIAGGPSSGAPPPSGGGGSGGGGSGGGGSQIVLTQSPAIMSASPGEKVTMTCSASSRVTYMHWYQQRSGTSPKRWIYDTSKLASGVPARFSGSGSGTSYSLTISSMEAEDAATYYCQQWGNNPQYTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 448 aa, synthetic construct
DVQLVESGGGLVQPGGSRKLSCAASGFTFSSFGMHWVRQAPEKGLEWVAYISSGSSTIYYADTVKGRFTISRDNPKNTLFLQMTSLRSEDTAMYYCARGKYPTWIAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 43 - protein, 450 aa, synthetic construct
QVTLKESGPGILQPSQTLSLTCSFSGFSLSTSGTGVGWIRQPSGKGLEWLSHIWWDDVKRYNPALKSRLTISRDTSYSQVFLRIASVDTADTATYYCARILDGTGPMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 408 aa, synthetic construct
QVQLVETGGGLVQAGGSLRLSCAASGRTFSSYRMGWFRQAPGKEREFVAVISRSGDVTTYAHSVKGRFTVSRDNAKNTVYLQMNSLKPEDTAVYYCAAGLAANTANYWGQGTQVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 45 - protein, 410 aa, synthetic construct
EVQLQESGGGLVQAGGSLRLSCAASGSISSLNGIWYRQAPGKQREVVAGITSGGSTNYADSVKGRFTISRDNAKNTVYLQMNGLKPEDTAIYYCNTVRNWDIRTALSRSGGQGTQVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 46 - protein, 408 aa, synthetic construct
EVQVQESGGGLVQAGGSLRLSCAASGRSFRSDRMGWFRQAPGKEREFVAVISRGGGVTTYADSVKGRFTVSRDDAKNTVDLQMNSLKPEDTAVYYCAAGITTKAANYWGMGTQVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 47 - protein, 405 aa, synthetic construct
QVQLVESGGGLVQPGGSLRLSCAASGSVFSRNFMGWYRQAPGKQRELVAYITVDGGTRYADSVKGRFTISRDNAKNTVYLQMNSLKPEDTAVYYCNRAGTTPYAWGQGTLVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 48 - protein, 411 aa, synthetic construct
QVRLVESGGGLVQAGGSLRLSCTASGRTFSSYSVGWFRQAPGKERAFVAVISKSGGITTYADSVKGRFIISRDNAKNTVYLQMNSLKPEDTAVYYCAASGSSLRLADSGSWGQGTQVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 49 - protein, 408 aa, synthetic construct
EVQLVESGGGLVQAGGSLRLSCVASGRTFGNYRMAWFRQAPGKEREFVAVISRNGGITTYADSAKGRFTLSRDNAKNAVYLQMNSLKPEDTAVYYCTGGRNLGTWDYWGQGTQVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 50 - protein, 408 aa, synthetic construct
QVQLVESGGGLVQAGGSLRLSCVASGRTFSSGRMAWFRQAPGKEREFVAVISQNGGVTTYAASVKGRFTVSRDNAKNTVYLQMNSLKPEDTAVYYCAAGVTTNTANYWGQGTRVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - protein, 414 aa, synthetic construct
EVQLQESGGGLVQAGGSLRLSCVVSGRTFGSLRMAWFRQAPGKEREFVSIISKSGGITTYADSVKGRFTISRDNAKNTVYLQMNSLKPEDTAVYYCAADASIRPRTNPSDYDGWGQGTQVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 52 - protein, 408 aa, synthetic construct
QVQLVESGGGLVQPGGSLRLSCVASGNIFSSSIMGWYRQAPGKQREAVAFITNGGSTDYADSVKGRFTISRDNAKNTTYLQMNSLKFEDTAVYYCNLRNQRNFQEGSLGQGTQVTVSSGGGGSGGGGSGGGGSHGEGTFTSDVSSYLEEQAAKEFIAWLVKGGGGGGGSGGGGSGGGGSAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 53 - protein, 402 aa, synthetic construct
EVQLVESGGGLVQAGGSLRLSCAASGIDFRADVMAWYRQAPGKQREVVAAITAYGDTDYVDSVKGRFTISRDNAKNTVYLLMNSLKPEDTAVYYCNVPVRRYWGQGTQVTVSSGGGGSGGGGSGGGGSYGEGTFTSDYSIYLDKIAQKAFVQWLIAGGPSSGAPPPSGGGGSGGGGSGGGGSDIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 407 aa, synthetic construct
EVQVVESGGGLVQPGGSLRLSCAASGSYFSINVMAWYRQAPGKQREVVAAISRGGFADYAQFAKGRFTISGDNAKNTVSLQMNSLKPEDTAVYYCNARRAWDTAINYWGQGTQVTVSSGGGGSGGGGSGGGGSYGEGTFTSDYSIYLDKIAQKAFVQWLIAGGPSSGAPPPSGGGGSGGGGSGGGGSDIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 411 aa, synthetic construct
QVQLVESGGGLVQPGGSLRLSCAASGSIHSINILGWYRQAPGDHRELVAGITTGGSTDYADSVKDRFTISRDNAKNTVYLQMNDLKPADTAVYYCNLRQSSTRLTWRPYDSWGQGTQVTVSSGGGGSGGGGSGGGGSYGEGTFTSDYSIYLDKIAQKAFVQWLIAGGPSSGAPPPSGGGGSGGGGSGGGGSDIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 411 aa, synthetic construct
QVQLVESGGGLVQPGGSLRLSCAASGFTFSSTSMTWVRQRPGRGLEWVSSIFSDGRSTYYADSLKGRVTISRDNAENTLYLQMNSLQSEDTAVYYCAKGLPSLRVGVMGLLRGQGTLVTVSSGGGGSGGGGSGGGGSYGEGTFTSDYSIYLDKIAQKAFVQWLIAGGPSSGAPPPSGGGGSGGGGSGGGGSDIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 57 - protein, 402 aa, synthetic construct
EVRLVESGGGLVQAGGSLRLSCAASGISFRADVMAWYRQAPGKQREVVAAITSYGDADYADSVKGRFTISRDNAKNTVYLQMNSLKPEDTAVYYCNVPARRYWGQGTQVTVSSGGGGSGGGGSGGGGSYGEGTFTSDYSIYLDKIAQKAFVQWLIAGGPSSGAPPPSGGGGSGGGGSGGGGSDIVMTQSPSSLTVTAGEKVTMSCKSSQSLLNSGNQKNYLTWYQQKPGQPPKLLISWASTRDSGVPDRFTGSGSGTDFTLTISSVQAEDLAVYYCQNDYSYPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 58 - protein, 118 aa, synthetic construct
QVQLVETGGGLVQAGGSLRLSCAASGRTFSSYRMGWFRQAPGKEREFVAVISRSGDVTTYAHSVKGRFTVSRDNAKNTVYLQMNSLKPEDTAVYYCAAGLAANTANYWGQGTQVTVSS

SEQ ID NO: 59 - protein, 120 aa, synthetic construct
EVQLQESGGGLVQAGGSLRLSCAASGSISSLNGIWYRQAPGKQREVVAGITSGGSTNYADSVKGRFTISRDNAKNTVYLQMNGLKPEDTAIYYCNTVRNWDIRTALSRSGGQGTQVTVSS

SEQ ID NO: 60 - protein, 120 aa, synthetic construct
QVQLVESGGGLVQPGGSLRLSCAASGSIFAINVMGWYRQASGNQRELVAGITTGGARDYADSVKGRFTISRDNAKNTVYLSMNSLKPEDTAVYYCNLHAPKYIDSRYDYWGQGTQVTVSS

SEQ ID NO: 61 - protein, 118 aa, synthetic construct
EVQVQESGGGLVQAGGSLRLSCAASGRSFRSDRMGWFRQAPGKEREFVAVISRGGGVTTYADSVKGRFTVSRDDAKNTVDLQMNSLKPEDTAVYYCAAGITTKAANYWGMGTQVTVSS

SEQ ID NO: 62 - protein, 121 aa, synthetic construct
QVRLVESGGGLVQAGGSLRLSCTASGRTFSSYSVGWFRQAPGKERAFVAVISKSGGITTYADSVKGRFIISRDNAKNTVYLQMNSLKPEDTAVYYCAASGSSLRLADSGSWGQGTQVTVSS

SEQ ID NO: 63 - protein, 118 aa, synthetic construct
EVQLVESGGGLVQAGGSLRLSCVASGRTFGNYRMAWFRQAPGKEREFVAVISRNGGITTYADSAKGRFTLSRDNAKNAVYLQMNSLKPEDTAVYYCTGGRNLGTWDYWGQGTQVTVSS

SEQ ID NO: 64 - protein, 118 aa, synthetic construct
QVQLVESGGGLVQAGGSLRLSCVASGRTFSSGRMAWFRQAPGKEREFVAVISQNGGVTTYAASVKGRFTVSRDNAKNTVYLQMNSLKPEDTAVYYCAAGVTTNTANYWGQGTRVTVSS

SEQ ID NO: 65 - protein, 124 aa, synthetic construct
EVQLQESGGGLVQAGGSLRLSCVVSGRTFGSLRMAWFRQAPGKEREFVSIISKSGGITTYADSVKGRFTISRDNAKNTVYLQMNSLKPEDTAVYYCAADASIRPRTNPSDYDGWGQGTQVTVSS

SEQ ID NO: 66 - protein, 118 aa, synthetic construct
QVQLVESGGGLVQPGGSLRLSCVASGNIFSSSIMGWYRQAPGKQREAVAFITNGGSTDYADSVKGRFTISRDNAKNTTYLQMNSLKFEDTAVYYCNLRNQRNFQEGSLGQGTQVTVSS

SEQ ID NO: 67 - protein, 113 aa, synthetic construct
EVQLVESGGGLVQAGGSLRLSCAASGIDFRADVMAWYRQAPGKQREVVAAITAYGDTDYVDSVKGRFTISRDNAKNTVYLLMNSLKPEDTAVYYCNVPVRRYWGQGTQVTVSS

SEQ ID NO: 68 - protein, 118 aa, synthetic construct
EVQVVESGGGLVQPGGSLRLSCAASGSYFSINVMAWYRQAPGKQREVVAAISRGGFADYAQFAKGRFTISGDNAKNTVSLQMNSLKPEDTAVYYCNARRAWDTAINYWGQGTQVTVSS

SEQ ID NO: 69 - protein, 122 aa, synthetic construct
QVQLVESGGGLVQPGGSLRLSCAASGFTFSSTSMTWVRQRPGRGLEWVSSIFSDGRSTYYADSLKGRVTISRDNAENTLYLQMNSLQSEDTAVYYCAKGLPSLRVGVMGLLRGQGTLVTVSS

SEQ ID NO: 70 - protein, 113 aa, synthetic construct
EVRLVESGGGLVQAGGSLRLSCAASGISFRADVMAWYRQAPGKQREVVAAITSYGDADYADSVKGRFTISRDNAKNTVYLQMNSLKPEDTAVYYCNVPARRYWGQGTQVTVSS

(19) United States
(12) Patent Application Publication
(10) Pub. No.: US 2025/0270274 A1
(43) Pub. Date: Aug. 28, 2025

(54) SUPER LONG-LASTING GLP1 OR GLP1/GIP ANALOGUE DRUG FOR TYPE-2 DIABETES AND OBESITY

(71) Applicant: Serpentide Inc., Hangzhou City (CN)

(72) Inventor: Yang WEI, Yortown Heights, NY (US)

(21) Appl. No.: 19/060,067

(22) Filed: Feb. 21, 2025

Related U.S. Application Data
(60) Provisional application No. 63/558,021, filed on Feb. 26, 2024.

(51) Int. Cl. C07K 14/605; A61K 38/00; A61K 47/68; A61P 3/04; A61P 3/10; C07K 16/26

(52) U.S. Cl. CPC C07K 14/605; A61K 47/6847; A61K 47/6889; A61P 3/04; A61P 3/10; C07K 16/26; A61K 38/00; C07K 2317/31; C07K 2317/569; C07K 2317/76; C07K 2319/50

(57) ABSTRACT

The present disclosure provides a GLP1 analogue and GLP1/GIP receptor co-agonist analogues, a fusion protein comprising the GLP1 analogue or the GLP1/GIP receptor co-agonist analogue, and methods of use thereof. In various embodiments of the invention, the fusion protein comprises the GLP1 analogue or the GLP1/GIP receptor co-agonist analogue fused to a protecting antibody or further fused to a stabilizing domain. In some embodiments, the fusion proteins are useful for treating or ameliorating a symptom or indication of a disorder such as obesity and diabetes.

### CROSS-REFERENCE TO RELATED APPLICATIONS

The present application claims the benefit of U.S. provisional application No. 63/558,021, filed on Feb. 26, 2025, which is incorporated herein in its entirety.

### REFERENCE TO SEQUENCE LISTING SUBMITTED ELECTRONICALLY

This application contains a sequence listing, which is submitted electronically as a XML formatted sequence listing with a file name “689339_32US_Sequence_Listing”, creation date of Jan. 20, 2025, and having a size of 81,417 bytes. The sequence listing submitted electronically is part of the specification and is herein incorporated by reference in its entirety.

### TECHNICAL FIELD

The present disclosure is related to human glucagon-like peptide 1 (GLP1) or GLP1/glucose-dependent insulinotropic polypeptide (GIP) analogue drugs, and to therapeutic methods of using said drugs.

### BACKGROUND

Obesity has become a major health issue in the United States. More than 40% of Americans are considered to be overweight or obese by modern standards due to poor diet, lack of exercise or other unhealthy behaviors. Obesity is an important underlying risk factor for developing other diseases such as heart disease, stroke, and diabetes. Typically, a modest decrease in excess body weight decreases the risk of developing certain obesity-associated diseases such as heart disease and diabetes.

Diabetes mellitus is a disorder in which the body does not produce enough insulin or respond normally to insulin, causing blood sugar level to be abnormally high. Long term levels of high blood sugar often result in undesirable long-term health consequences including heart disease, stroke, poor overall circulation, and in severe cases, lower limb amputation. Treatment of diabetes usually involves controlling and/or reducing blood sugar levels through a combination of regular exercise and dietary control along with certain medications such as insulin and/or metformin.

Mature glucagon-like peptide 1 (GLP1(7-37) with sequence HAEGTFTSDVSSYLEGQAAKEFIAWLVKGRG (SEQ ID NO: 1) is a 31-amino acid peptide hormone released from intestinal “L” cells following nutrient consumption. It potentiates the glucose-induced secretion of insulin from pancreatic beta cells, increases insulin expression, inhibits beta cell apoptosis, promotes beta cell neogenesis, reduces glucagon secretion, delays gastric emptying, promotes satiety and increases peripheral glucose disposal. These multiple effects have generated great interest in identifying long-lasting agonists of the GLP1 receptor (GLP1R) in order to treat Type 2 diabetes.

One of the approaches used for treating diabetes and for glycemic control involves the use of GLP-1R agonists that target the incretin pathway. GLP1 exerts its main effect by stimulating glucose-dependent insulin release from the pancreatic islets. It has also been shown to slow gastric emptying, inhibit inappropriate post-meal glucagon release and reduce food intake. However, GLP1 is quickly inactivated and/or degraded by the enzyme such as dipeptidyl peptidase 4 (DPP4), leading to a very short half-life of about 1.5 minutes. This is undesirable as it severely limits its effectiveness in regulating blood sugar levels. Longer-acting derivatives of GLP1 as well as GLP1 receptor agonists including fusion proteins including GLP1 provide an improved incretin effect, and therefore have been studied for diabetes control. Such GLP1 analogues, fusion proteins and GLP1 receptor agonists are well known in the art, with exemplary examples disclosed, for example, in U.S. Pat. No. 9,409,966, and published applications including U.S. 20160194371, U.S. 20170114115, U.S. 20170112904, U.S. 20160361390, U.S. 20150259416, WO2017074715, WO2016127887, EP3034514, EP2470198, and EP2373681.

GLP1 peptide variants and GLP1 receptor agonists that are resistant to degradation by DPP4 and other factors have also been explored. Previous work has shown that various amino acid substitutions at position 8 of GLP1 (7-37) make such peptides more resistant to DPP4, thus conferring a longer half-life. Certain other GLP1 receptor agonists are also disclosed in U.S. 2019/0091296A1 to Wei et al. which is hereby incorporated by reference in its entirety. Although those variants provide the protection for the DPP4 cleavage at some degree, they only prolong the GLP1's half life to several hours in mice, and the GLP1 is still subject to DPP4 and other protease cleavage. Thus, there is a need to develop new molecules that are more resistant to DPP4 and other proteases.

Glucose-dependent insulinotropic polypeptide (GIP) is a 42-amino acid gastrointestinal regulatory peptide that plays a physiological role in glucose homeostasis by stimulating insulin secretion from pancreatic beta cells in the presence of glucose and protecting pancreatic bata cells. GIP and GLP1 are known as incretins; incretin receptor signaling exerts physiologically relevant action critical for glucose homeostasis. GIP alone has very modest glucose-lowering ability in Type 2 diabetic humans. Both native GIP and GLP1 are inactivated rapidly by the ubiquitous protease, DPP4, and therefore, can only be used for short-term metabolic control. Certain GIP analogs have been described as exhibiting both GIP and GLP1 activity in WO 2013/164483, WO 2014/192284, and WO 2011/119657. GLP1/GIP receptors co-agonists also have been disclosed in U.S. Pat. No. 9,474,780B2. Those GLP1/GIP receptor co-agonists have the same short half-life issues as GLP1, and are also subject to protease degradation.

Therefore, new molecules that are more resistant to DPP4 and other proteases and have better blood sugar and body weight loss effects need to be developed.

### SUMMARY

According to a first aspect, the present disclosure provides a glucagon-like peptide 1 (GLP1) analogue SP01 with the amino acid sequence of SEQ ID NO: 2. Compared with mature glucagon-like peptide 1 (GLP1 (7-37), the changes in the amino acid sequence include A8G and R36G. The present disclosure also provides a GLP1/GIP receptor co-agonist analogue SP02 or SP03, the amino acid sequences of SP02 and SP03 are SEQ ID NO: 4 and SEQ ID NO: 5, respectively. Compared with Tirzepatide (SEQ ID NO: 3), the changes in the amino acids involve replacing the unnatural amino acid α-aminoisobutyric acid at positions 2 and 30 with glycine and tyrosine, and glycine and glutamine, respectively. SP01, SP02 or SP03 can enhance resistance to proteolytic cleavage and confer a longer half-life. The fusion proteins containing SP01, SP02 and SP03 can be synthesized by CHO cells and easily purified.

According to a second aspect, the present disclosure provides a fusion protein including: a) GLP1 analogue SP01, GLP1/GIP receptor co-agonist analogue SP02 or GLP1/GIP receptor co-agonist analogue SP03; and b) a protecting antibody fused to the N-terminus of GLP1 analogue or GLP1/GIP receptor co-agonist analogue through a linker. The protecting antibody specifically binds to GLP1 analogue or GLP1/GIP receptor co-agonist analogue and protects it from proteolytic cleavage. The fusion protein can enhance resistance to proteolytic cleavage and confer a longer half-life. In such embodiments, the fusion protein is and remains inactive until the proteolytic cleavage removes the protecting antibody. In certain embodiments, the protecting antibody is a nanobody. In certain embodiments, the linker is the commonly used short amino acids sequence, preferably 3×G4S(GGGGSGGGGSGGGGS) followed by a proteolytic cleavage sequence. The preferred proteolytic cleavage sequence is Factor Xa and its variant (i.e., RGER, RKR, RGR, RSR, RR etc.). In a specific embodiment, the fusion protein includes: a) GLP1 analogue SP01; and b) a nanobody fused to the N-terminus of GLP1 analogue. The sequence of the nanobody is at least 90% identity, at least 95% identity, at least 99% identity with any one of SEQ ID NOs: 21, 58, 59 and 61 to 66. The sequence of the nanobody preferably is any one of SEQ ID NOs: 21, 58, 59 and 61 to 66, and more preferably is SEQ ID NO: 21. The sequence of the nanobody preferably is the humanized modified sequence of any one of SEQ ID NOs: 21, 58, 59 and 61 to 66, and more preferably is the humanized modified sequence of SEQ ID NO: 21. In a specific embodiment, the fusion protein includes: a) GLP1/GIP receptor co-agonist analogue SP02; and b) a nanobody fused to the N-terminus of GLP1/GIP analogue. The sequence of the nanobody is at least 90% identity, at least 95% identity, at least 99% identity with any one of SEQ ID NOs: 34, and 67 to 70. The sequence of the nanobody preferably is any one of SEQ ID NOs: 34, and 67 to 70, and more preferably is SEQ ID NO: 34. The sequence of the nanobody preferably is the humanized modified sequence of any one of SEQ ID NOs: 34, and 67 to 70, and more preferably is the humanized modified sequence of SEQ ID NO: 34.

In certain embodiments, the fusion protein described in the second aspect further includes a stabilizing domain fused to the C-terminus of GLP1 analogue or GLP1/GIP receptor co-agonist analogue through a linker. The stabilizing domain can be an Fc part, or an antibody or antigen-binding fragment thereof that specifically binds GLP1 receptor, or GIP receptor, or a GIP receptor blocking antibody or antigen-binding fragment thereof, or bispecific antibodies of GLP1 receptor and GIP receptor or antigen-binding fragment thereof, or a nanobody specifically binds to albumin, or an albumin-binding domain (ABD). When the stabilizing domain is an antibody, for example, an anti GIP receptor, it not only increases the stability of SP02, but also enhances the activity of SP02 towards GIP receptor because of the tethering effect. In specific embodiments, the GLP1 analogue or GLP1/GIP receptor co-agonist analogue is further fused to: c) the N-terminus of the Fc part of the immunoglobulin, preferably is from IgG4 or IgG2, or their variants; d) the N-terminus of the light chain or heavy chain of the antibody or antigen-binding fragment thereof that specifically binds to GLP1 receptor; c) the N-terminus of the light chain or heavy chain of the antibody or antigen-binding fragment thereof that specifically binds to GIP receptor, or specifically block GIPR activity; or f) the N-terminus of the light chain or heavy chain of a bispecific antibody or antigen-binding fragment thereof that specifically bind to both GLP1 receptor and GIP receptor; g) an N-terminus of a nanobody that specifically binds to albumin; h) an N-terminus of an albumin-binding domain (ABD) that specifically binds to albumin, at the C-terminus through a linker. The linker is preferably 3×G4S(GGGGSGGGGSGGGGS).

According to a third aspect, the present disclosure provides nucleic acid molecules encoding the fusion protein described in the second aspect. The present disclosure also provides recombinant expression vectors capable of expressing the fusion protein described in the second aspect. For example, the present disclosure includes recombinant expression vectors including any of the above nucleic acid molecules. Also included within the scope of the present disclosure are host cells into which such vectors have been introduced, as well as methods of producing the proteins or fragments thereof by culturing the host cells under conditions permitting production of the proteins or fragments thereof, and recovering the proteins and fragments so produced.

According to a fourth aspect, the present disclosure provides a pharmaceutical composition including a therapeutically effective amount of at least one agent selected from the fusion proteins described in the second aspect, and a medicinal carrier or excipient. The pharmaceutical composition may further include a second therapeutic agent. The present disclosure provides a therapeutic method for preventing, improving, or treating GLP1-related diseases or conditions (e.g., diabetes, obesity, Type 2 diabetes) in a subject, where the therapeutic method includes administering a therapeutically effective amount of at least one agent selected from the fusion proteins described in the second or third aspect, or the pharmaceutical composition described above. In certain embodiments, the present disclosure provides a method to reduce blood sugar levels or body weight in a subject, where the method includes administering a therapeutically effective amount of at least one agent selected from the fusion proteins described in the second or third aspect, or the pharmaceutical composition described above. The above methods may also include a combination with a second therapeutic agent or therapy.

### BRIEF DESCRIPTION OF THE DRAWINGS

The above and other objects and advantages of the present disclosure will be apparent upon consideration of the following detailed description, taken in conjunction with the accompanying drawings, in which like reference characters refer to like parts throughout, and in which:

FIG. 1A-FIG. 1H shows the pharmacokinetic profiles of P93 and P99 in C57BL mice after subcutaneous (SC) administration of 100 or 300 nmol/kg. All data are expressed as mean±SEM.

FIG. 2A and FIG. 2B shows the pharmacokinetic profiles of P95 in C57BL mice after subcutaneous (SC) administration of 100 or 300 nmol/kg. All data are expressed as mean±SEM.

FIG. 3A-FIG. 3I shows the IPGTT results in C57BL mice. Drugs were administered subcutaneously at 100 or 300 nmol/kg on Day 0. Intraperitoneal glucose tolerance test (IPGTT) was performed on Day 1 (A, E), 7 (B, F), 14 (C, G), 21 (D, H) and 28 (I) after 6 hours fasting with measurements of blood glucose concentrations at 0, 15, 30, 60, 90 and 120 minutes. All data are expressed as mean±SEM.

FIG. 4A-FIG. 4G shows the IPGTT results in C57BL mice. Drugs P46, P86 and P98 were administered subcutaneously at 30, 100 or 300 nmol/kg on Day 0. Intraperitoneal glucose tolerance test (IPGTT) was performed on Day 1 (A, D), 5 (B), 7 (E), 10 (C), 14 (F) and 21 (G) after 6 hours fasting with measurements of blood glucose concentrations at 0, 15, 30, 60, 90 and 120 minutes. All data are expressed as mean±SEM.

FIG. 5A-FIG. 5D shows the IPGTT results in C57BL mice. Drugs P19, P28 and P100 were administered subcutaneously at 15 nmol/kg for P19, 10 or 30 nmol/kg for P28, 60 nmol/kg for P100 on Day 0. Intraperitoneal glucose tolerance test (IPGTT) was performed on Day 1 (A and C), 7 (B and D), after 6 hours fasting with measurements of blood glucose concentrations at 0, 15, 30, 60, 90 and 120 minutes. All data are expressed as mean±SEM.

FIG. 6 shows weight loss in 18 weeks old DIO mice chronically administered the pharmaceutical product dulaglutide sold under the brand name TRULICITY® at 10 or 30 nmol/kg twice weekly; tirzepatide and P46 at 30 nmol/kg twice weekly; P99, P93, P98, P86, P28 at 100 nmol/kg once weekly; P100 at 60 nmol/kg once weekly. The starting body weight of DIO mice was around 45 g. P93 shows equivalent weight loss to dulaglutide (TRULICITY® brand) at 10 nmol/kg (12% vs. 11%), P99 shows better weight loss than dulaglutide (TRULICITY® brand) at 30 nmol/kg (20.7% vs. 16.6%).

FIG. 7A and FIG. 7B shows blood glucose (FIG. 7A) and cumulative food intake (FIG. 7B) in DIO mice chronically administered dulaglutide (TRULICITY® brand) at 10 and 30 nmol/kg twice weekly; tirzepatide and P46 at 30 nmol/kg twice weekly; P99, P93, P98, P86 at 100 nmol/kg once weekly. P93, P99, P86, P98 all show significant blood glucose control. P99 shows significant reduction of food intake like Tirzepatide.

### DETAILED DESCRIPTION

Before the present methods are described, it is to be understood that this invention is not limited to particular methods, or the experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present disclosure, preferred methods and materials are now described. All publications mentioned herein are incorporated herein by reference in their entirety.

As used herein, a “stabilizing domain” is any macromolecule that when fused to a peptide increases the in vivo activity and/or stability of the peptide. For example, a stabilizing domain may be a polypeptide including an immunoglobulin C_{H}3 domain. In certain embodiments, the stabilizing domain increases the serum half-life of the peptide. In certain embodiments, the stabilizing domain increases the in vivo potency of the peptide. A non-limiting example of a stabilizing domain is an Fc portion of an immunoglobulin, e.g., an Fc domain of an IgG selected from the isotypes IgG1, IgG2, IgG3, and IgG4, as well as any allotype within each isotype group. In certain embodiments, the stabilizing domain is an Fc fragment or an amino acid sequence of 1 to about 200 amino acids in length containing at least one cysteine residues. As another example, a stabilizing domain may be an immunoglobulin or an antigen binding fragment thereof. In certain embodiments, the stabilizing domain is an immunoglobulin including a heavy chain variable region and a light chain variable region wherein the immunoglobulin binds to a specific antigen. In certain embodiments, the stabilizing domain includes an antigen-binding domain and an Fc domain (for example, of an IgG1 or IgG4 antibody) or may include only an antigen-binding portion (for example, a Fab, F (ab′)2 or scFv fragment), and may be modified to affect functionality. In a specific embodiment, the stabilizing domain is an immunoglobulin including a heavy chain variable region and a light chain variable region wherein the immunoglobulin binds to GLP1 receptor and/or GIP receptor. In other embodiments, the stabilizing domain is a cysteine residue or a short cysteine-containing peptide. Other stabilizing domains include peptides or polypeptides including or consisting of a leucine zipper, a helix-loop motif, or a coiled-coil motif.

The term “antibody”, as used herein, is intended to refer to immunoglobulin molecules including four polypeptide chains, two heavy (H) chains and two light (L) chains inter-connected by disulfide bonds, as well as multimers thereof (e.g. IgM) or antigen-binding fragments thereof. Each heavy chain includes a heavy chain constant region (including domains C_{H}l, C_{H}2, and C_{H}3) and an lg variable region which may be a heavy chain variable region (“HCVR” or “V_{H}”) or a light chain variable region (“LCVR” or “V_{L}”). Each light chain includes a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy terminus in the following order: FR1, CDR1, FR2, CDR2, FR3, CDR3, FR4. In certain embodiments of the invention, the FRs of the antibody (or antigen-binding fragment thereof) may be identical to the human germline sequences, or may be naturally or artificially modified. An amino acid consensus sequence may be defined based on a side-by-side analysis of two or more CDRs. The term “antigen-binding protein”, as used herein, also includes antibodies.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

Methods and techniques for identifying CDRs within VR amino acid sequences are well known in the art and can be used to identify CDRs within the specified VR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within the antigen-binding domain of an antigen-binding protein or an antibody.

CDR residues not contacting antigen can be identified based on previous studies (for example, residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

The term “antigen-binding fragment” as used herein, includes any naturally occurring, enzymatically obtainable, synthetic, or genetically engineered polypeptide or glycoprotein that specifically binds an antigen to form a complex. The term “antigen-binding fragment” as used herein, refers to one or more fragments of an antigen-binding protein that retain the ability to specifically bind to GLP1 receptor or GIP receptor. An antigen-binding fragment may include a Fab fragment, a F(ab′)2 fragment, a Fv fragment, a dAb fragment, a fragment containing a CDR, or an isolated CDR. In certain embodiments, the term “antigen-binding fragment” refers to a polypeptide fragment of a multi-specific antigen-binding molecule. Antigen-binding fragments may be derived, e.g., from full protein molecules using any suitable standard techniques such as proteolytic digestion or recombinant genetic engineering techniques involving the manipulation and expression of DNA encoding antigen-binding protein variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment” as used herein.

An antigen-binding fragment of the present disclosure will typically include at least one immunoglobulin (Ig) variable domain. The variable domain may be of any size or amino acid composition and will generally include at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L}, or V_{L}-V_{L}, dimers. Alternatively, the antigen-binding fragment of an antigen binding protein may contain a monomeric V_{H} or V_{L} domain.

In certain embodiments, an antigen-binding fragment may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of the present disclosure may include a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full protein molecules, antigen-binding fragments may be mono-specific or multi-specific (e.g., bi-specific). A multi-specific antigen-binding fragment will typically include at least two different variable domains, where each variable domain is capable of specifically binding to a separate antigen or to a different epitope on the same antigen. Any multi-specific antigen-binding protein format, including the exemplary bi-specific antigen-binding protein formats disclosed herein, may be adapted for use in the context of an antigen-binding fragment of the present disclosure using routine techniques available in the art.

The term “a protecting antibody” as used herein, includes an antibody's Fab region, a nanobody and bispecific T-cell engager “BiTE” antibody.

The term “nanobody” as used herein is a new type of antibody with the characteristics of small relative molecular weight, good stability, strong solubility, good antigen binding performance and low immunogenicity. There is a natural heavy chain antibody with a missing light chain in the Camelidae, and cloning its variable region can obtain a single-domain antibody composed of only a heavy chain variable region, called VHH (Variable domain of heavy chain antibody), also known as nanobody (a single domain antibody), which is the smallest functional antigen-binding fragment. Compared with ordinary antibodies, nanobodies have small molecular weight, simple structure, easy genetic modification, small size, good antigen specificity, strong tissue penetration, and high stability. The nanobodies by humanized modification have more than 90%, or more than 95%, or more than 99% homology.

To confer better resistance to proteolytic cleavage, a first step was to change the sequence of GLP1 or GLP1/GIP1 receptor co-agonist to provide better resistance to proteolytic cleavage. The present disclosure has shown herein that the novel GLP1 analogue or GLP1/GIP1 receptor co-agonist analogue is indeed highly resistant to degradation by protease. Optionally, it is further fused to a protecting antibody such as a nanobody, which can be specifically bound to it, through a linker at its N-terminus, and then the resistance to proteolytic cleavage has been further enhanced. A second step was to compensate for any weakened or reduced GLP1 activity by fusing the GLP1 analogue or GLP1/GIP1 receptor co-agonist analogue to the corresponding stabilizing domain, which tethers the weakened GLP1 or GLP1/GIP1 receptor co-agonist to the receptor and thereby increases its potency. Further, the present disclosure found that these fusion proteins have a prolonged serum half-life and result in a decrease in blood glucose levels that lasts longer.

The GLP1 analogue or GLP1/GIP1 receptor co-agonist analogue, or the fusion protein thereof of the present disclosure are useful for the treatment, and/or prevention of a disease or disorder or condition associated with hyperglycemia such as diabetes and/or for ameliorating at least one symptom associated with such disease, disorder or condition. In one embodiment, the GLP1 analogue or GLP1/GIP1 receptor co-agonist analogue, or the fusion protein thereof of the present disclosure may be administered at a therapeutic dose to a patient with diabetes (e.g., Type 2 diabetes).

In certain embodiments, the GLP1 analogue or GLP1/GIP1 receptor co-agonist analogue, or the fusion protein thereof of the present disclosure are useful to treat subjects suffering from a disease or disorder selected from the group consisting of diabetes mellitus, obesity, insulin resistance, hypertension, dyslipidemia, Type 2 diabetes, Type 1 diabetes, prediabetes, cardiovascular disease, atherosclerosis, congestive heart failure, coronary heart disease, arteriosclerosis, peripheral artery disease, stroke, respiratory dysfunction, renal disease, fatty liver disease, non-alcoholic steatohepatitis (NASH), and metabolic syndrome.

In certain embodiments, the GLP1 analogue or GLP1/GIP1 receptor co-agonist analogue, or the fusion protein thereof of the present disclosure are useful to treat subjects that are overweight or obese and/or prevent or treat one or more obesity-associated disorders such as heart disease, stroke, and diabetes.

In certain embodiments, the GLP1 analogue or GLP1/GIP1 receptor co-agonist analogue, or the fusion protein thereof of the present disclosure are useful to treat subjects suffering from diabetes and/or prevent one or more complications of diabetes such as heart disease, stroke, kidney disease, retinopathy, blindness and nerve damage.

It is also contemplated herein to use one or more selected from the GLP1 analogue or GLP1/GIP1 receptor co-agonist analogue, or the fusion protein thereof of the present disclosure prophylactically to subjects at risk for developing diabetes (e.g., Type 2 diabetes). The subjects at risk include, but are not limited to, subjects of advanced age, pregnant women, and subjects with one or more risk factors including family history of obesity, high blood cholesterol, smoking, excessive alcohol consumption, and/or lack of exercise.

In a further embodiment, the GLP1 analogue or GLP1/GIP1 receptor co-agonist analogue, or the fusion protein thereof of the present disclosure are used for the preparation of a pharmaceutical composition or medicament for treating patients suffering from a disease or disorder such as diabetes and obesity. In another embodiment of the invention, the GLP1 analogue or GLP1/GIP1 receptor co-agonist analogue, or the fusion protein thereof of the present disclosure are used as adjunct therapy with any other agent or any other therapy known to those skilled in the art useful for treating or ameliorating a disease or disorder associated with hyperglycemia such as diabetes (e.g., Type 2 diabetes).

Combination therapies may include the GLP1 analogue or GLP1/GIP1 receptor co-agonist analogue, or the fusion protein thereof of the present disclosure and any additional therapeutic agent that may be advantageously combined with the GLP1 analogue or GLP1/GIP1 receptor co-agonist analogue, or the fusion protein thereof. The GLP1 analogue or GLP1/GIP1 receptor co-agonist analogue, or the fusion protein thereof of the present disclosure may be combined synergistically with one or more drugs or therapy used to treat any disease or disorder associated with hyperglycemia (e.g., diabetes). In some embodiments, the GLP1 analogue or GLP1/GIP1 receptor co-agonist analogue, or the fusion protein thereof of the present disclosure may be combined with a second therapeutic agent to reduce blood sugar levels in a subject, or to ameliorate one or more symptoms of diabetes.

The GLP1 analogue or GLP1/GIP1 receptor co-agonist analogue, or the fusion protein thereof of the present disclosure may be used in combination with insulin (or an insulin analog), insulin sensitizers such as biguanides (e.g., metformin), and thiazolidinediones (e.g., rosiglitazone), insulin secretagogues such as sulphonylureas (e.g., chlorpropamide), and glinides (e.g., nateglinide), alpha-glucosidase inhibitors (e.g., acarbose), dipeptidyl peptidase 4 (DPP4) inhibitors (e.g., sitagliptin), pramlinitide, bromocriptine, sodium glucose cotransporter 2 (SGLT-2) inhibitors (e.g., canagliflozin), an anti-hypertensive drug (e.g., an angiotensin-converting enzyme inhibitor, an angiotensin receptor blocker, a diuretic, a calcium channel blocker, an alpha-adrenoceptor blocker, an endothelin-receptor blocker, an organic nitrate, and a protein kinase C inhibitor), a statin, aspirin, a different GLP1 receptor agonist, a different GLP1/GIP receptor co-agonist, a dietary supplement or any other therapy (e.g., exercise) to treat or manage diabetes. In certain embodiments, The GLP1 analogue or GLP1/GIP1 receptor co-agonist analogue, or the fusion protein thereof of the present disclosure may be administered in combination with a second therapeutic agent or therapy selected from the group consisting of insulin, an insulin analog, metformin, rosiglitazone, pioglitazone, chlorpropamide, glibenclamide, glimepiride, glipizide, tolazamide, tolbutamide, nateglinide, repaglinide, acarbose, miglitol, exenatide, liraglutide, albiglutide, dulaglutide, sitagliptin, saxagliptin, linagliptin, alogliptin, pramlinitide, bromocriptine quick-release, canagliflozin, dapagliflozin, empagliflozin, diet modifications and exercise.

As used herein, the term “in combination with” means that additional therapeutically active component(s) may be administered prior to, concurrent with, or after the administration of the GLP1 analogue or GLP1/GIP1 receptor co-agonist analogue, or the fusion protein thereof of the present disclosure.

The present disclosure includes pharmaceutical compositions in which the GLP1 analogue or GLP1/GIP1 receptor co-agonist analogue, or the fusion protein thereof of the present disclosure is co-formulated with one or more of the additional therapeutically active component(s) as described elsewhere herein.
For example, in Embodiment 1, the present disclosure provides a glucagon-like peptide 1 (GLP1) analogue SP01 with the amino acid sequence as SEQ ID NO: 2, a GLP1/GIP receptor co-agonist analogue SP02 or SP03, the amino acid sequences of SP02 and SP03 are SEQ ID NO: 4 and SEQ ID NO: 5.
In Embodiment 2, the present disclosure provides a fusion protein including: a) GLP1 analogue SP01, GLP1/GIP receptor co-agonist analogue SP02 or GLP1/GIP receptor co-agonist analogue SP03; and b) a protecting antibody fused to the N-terminus of GLP1 analogue or GLP1/GIP receptor co-agonist analogue through a linker. The protecting antibody specifically binds to GLP1 analogue or GLP1/GIP receptor co-agonist analogue and protects it from proteolytic cleavage. The fusion protein can enhance resistance to proteolytic cleavage and confer a longer half-life.
In Embodiment 3, the present disclosure provides the fusion protein of embodiment 2, where the protecting antibody is a nanobody.
In Embodiment 4, the present disclosure provides the fusion protein of embodiment 3, where the linker is 3×G4S(GGGGSGGGGSGGGGS) followed by a proteolytic cleavage sequence, the preferred proteolytic cleavage sequence is Factor Xa and its variant (i.e. RGER, RKR, RGR, RSR, RR etc.).
In Embodiment 5, the present disclosure provides the fusion protein of any of embodiment 3-4, where the fusion protein includes: a) GLP1 analogue SP01; and b) a nanobody fused to the N-terminus of GLP1 analogue. The sequence of the nanobody is at least 90% identity, at least 95% identity, at least 99% identity with any one of SEQ ID NOs: 21, 58, 59 and 61 to 66. The sequence of the nanobody preferably is any one of SEQ ID NOs: 21, 58, 59 and 61 to 66, and more preferably is SEQ ID NO: 21.
In Embodiment 6, the present disclosure provides the fusion protein of any of embodiment 3-4, where the fusion protein includes: a) GLP1/GIP receptor co-agonist analogue SP02; and b) a nanobody fused to the N-terminus of GLP1/GIP analogue, the sequence of the nanobody is at least 90% identity, at least 95% identity, at least 99% identity with any one of SEQ ID NOs: 34, and 67 to 70. The sequence of the nanobody preferably is any one of SEQ ID NOs: 34, and 67 to 70, and more preferably is SEQ ID NO: 34.
In Embodiment 7, the present disclosure provides the fusion protein of any of embodiment 2-6, where GLP1 analogue or GLP1/GIP receptor co-agonist analogue is further fused to a stabilizing domain at the C-terminus through a linker.
In Embodiment 8, the present disclosure provides the fusion protein of embodiment 7, where the GLP1 analogue or GLP1/GIP receptor co-agonist analogue is further fused to: c) the N-terminus of the Fc part of the immunoglobulin, preferably is from IgG4 or IgG2, or their variants; or d) the N- terminus of the light chain or heavy chain of the antibody or antigen-binding fragment thereof that specifically binds to GLP1 receptor; or e) the N-terminus of the light chain or heavy chain of the antibody or antigen-binding fragment thereof that specifically binds to GIP receptor, or specifically block GIPR activity; or f) the N-terminus of the light chain or heavy chain of a bispecific antibody or antigen-binding fragment thereof that specifically bind to both GLP1 receptor and GIP receptor, at the C-terminus through a linker.
In Embodiment 9, the present disclosure provides the fusion protein of embodiment 13 or 14, the stabilizing domain fuses to the GLP1 analogue or GLP1/GIP receptor co-agonist analogue through a linker, preferably 3×G4S(GGGGSGGGGSGGGGS).
In Embodiment 16, the present disclosure provides the fusion protein of embodiment 7 or 8, where the linker is preferably 3×G4S(GGGGSGGGGSGGGGS).
In Embodiment 10, the present disclosure provides nucleic acid molecules encoding the fusion protein of any of embodiment 2-9.
In Embodiment 11, the present disclosure provides recombinant expression vectors capable of expressing the fusion protein of any of embodiment 2-9.
In Embodiment 12, the present disclosure provides a pharmaceutical composition including a therapeutically effective amount of at least one agent selected from the fusion protein of any of embodiment 2-9, and a medicinal carrier or excipient, the pharmaceutical composition may further include a second therapeutic agent.
In Embodiment 13, the present disclosure provides a therapeutic method for prevent, improve or treat GLP1-related diseases or conditions (e.g., diabetes, obesity, Type 2 diabetes) in the subject, where the therapeutic method includes administering a therapeutically effective amount of at least one agent selected from the fusion protein of any of embodiment 2-9 or the pharmaceutical composition according to claim 12.
In Embodiment 14, the present disclosure provides a method to reduce blood sugar levels or reduce body weight in the subject, the method includes administering a therapeutically effective amount of at least one agent selected from the fusion protein of any of embodiment 2-9 or the pharmaceutical composition according to claim 12.
In Embodiment 15, the present disclosure provides the method of embodiment 13 or 14, the method may also include a combination with a second therapeutic agent or therapy.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.), but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Reagents Used and Lot Numbers

| | 1. SP01: exemplary GLP1 |
|---|---|
| | analogue |
| (SEQ ID NO: 2) | |
| | HGEGTFTSDVSSYLEEQAAKEFIAWLVKGGG. |
| | 2. SP02: GLP1/GIP receptor |
| | co-agonist analogue |
| (SEQ ID NO: 4) | |
| | YGEGTFTSDYSIYLDKIAQKAFVQWLIAGGPSSGAPPPS. |
| | 3. SP03: GLP1/GIP receptor |
| | co-agonist analogue |
| (SEQ ID NO: 5) | |
| | YGEGTFTSDYSIQLDKIAQKAFVQWLIAGGPSSGAPPPS. |

4. Eli Lilly's dulaglutide pharmaceutical product, sold under the brand name TRULICITY®: manufactured by Vetter Pharma-Fertigung GmbH & Co.KG, Lilly France.
5. P19: Eli Lilly's Tirzepatide synthesized by ZHEJIANG PEPTITES BIOTECH CO., LTD, China, Cat #603733.
6. P26 (SEQ ID NO: 40-43): SP02-bispecific antibodies for GLP1R and GIPR.
7. P28 (SEQ ID NO: 38-39): SP02-amG2
8. P46 (SEQ ID NO: 29-27): SP01-amG3.
9. (P62-P73)-hIgG1Fc (SEQ ID NO: 6-15): 10 SP01 nanobodies fused to hIgG1Fc.
10. (P49-59)-hIgG1Fc (SEQ ID NO: 16-20): 5 SP02 nanobodies fused to hIgG1Fc.
11. P86 (SEQ ID NO: 26-27): P67-RGER-SP01-amG3.
12. P93 (SEQ ID NO: 22): P67-RGER-SP01-hIgG4Fc.
13. P95 (SEQ ID NO: 35-36): P56-RGER-SP02-amG2.
14. P98 (SEQ ID NO: 28-27): P67-RKR-SP01-amG3.
15. P99 (SEQ ID NO: 23): P67-RKR-SP01-hIgG4Fc.
16. P100 (SEQ ID NO: 37-36): P56-RKR-SP02-amG2.

### Example 1: Design of GLP 1 Analogue and GLP1/GIP Receptor Co-Agonist Analogue

Mature GLP1 is a 31-amino acid peptide hormone that includes amino acids 7 to 37 of full-length GLP1 and has the amino acid sequence: HAEGTFTSDVSSYLEGQAAKEFIAWLVKGRG (SEQ ID NO: 1).

Mature GLP1 was modified by amino acid mutations to generate GLP1 analogues. The exemplary GLP1 analogue SP01 has the amino acid sequence: HGEGTFTSDVSSYLEEQAAKEFIAWLVKGGG (SEQ ID NO: 2). Compared with mature GLP1, the changes to the amino acid sequence are A8G, G22E and R36G.

GLP1/GIP screen: Eli Lily's Tirzepatide (SEQ ID NO: 3) is a GLP1/GIP receptor dual agonist and has the amino acid sequence: Y[Aib]EGTFTSDYSI[Aib]LDKIAQK[diacid-gamma-Glu-(AEEA)2]AFVQWLIAGGPSSGAPPPS(SEQ ID NO: 3).

Two new GLP1/GIP receptor co-agonists were obtained by changing the two non-coding amino acid residues at positions 2 and 13 (α-aminoisobutyric acid) to glycine and tyrosine or glutamine, respectively.

The exemplary GLP1/GIP receptor co-agonist analogues SP02, SP03 have the amino acid sequences as follows:

SP02 (SEQ ID NO: 4):
YGEGTFTSDYSIYLDKIAQKAFVQWLIAGGPSSGAPPPS,
SP03 (SEQ ID NO: 5):
YGEGTFTSDYSIQLDKIAQKAFVQWLIAGGPSSGAPPPS.

### Example 2: HTRF CAMP bioassay for GLP1/GIP receptor co-agonist analogues

The GLP1/GIP receptor co-agonist analogues were tested for their ability to stimulate cAMP production in HEK293/hGLP1R/CRE-LUC and HEK293/hGIPR/CRE-LUC cell lines.

For the homogeneous time-resolved fluorescence (HTRF) CAMP bioassay (Cisbio, Cat #62AM4PEJ), the cells were grown in DMEM 90%, FBS 10%, hygromycin B 100 μg/mL at 37° C., 5% CO₂, and then seeded into a 384-well assay plate (Greiner, Cat #784075) containing Assay buffer for DPBS (0.5 mM IBMX and 0.1% BSA) at 1000 cells/well.

To determine the dose-response of the test peptides, human GLP1, human GIP, dulaglutide (TRULICITY® brand), P19, SP02, SP03 were added to the cells at concentrations ranging from 0.02 pM to 100 nM. After 30 minutes of incubation at 37° C., 5 μL cAMP Eu-cryptate working solution (Promega) and 5 μL Anti cAMP-d2 working solution were added to each well of the assay plate. Cover the plate and incubate for 1 h at room temperature, and read the fluorescence at 665 and 620 nm with an EnVision plate reader (PerkinElmer) with a time-resolved fluorescence laser (TRF LASER). The results were analyzed using nonlinear regression (three parameters) with Prism 6 software (GraphPad) to obtain EC₅₀ values.

As shown in Table 1, SP02 and SP03 show EC₅₀ values of 0.024 nM and 0.225 nM, respectively, for GLP1R activation, and 3.69 nM and 22 nM, respectively, for GIPR activation. SP02 has an EC₅₀ superior to SP03 and was selected for the subsequent study.

TABLE 1
EC₅₀ for GLP1/GIP receptor co-agonist analogues

| | | |
|---|---|---|
| Cell Line | hGLP1R | hGIPR |

| | | | | |
|---|---|---|---|---|
| Tirzepatide | 0.20 | nM | 0.19 | nM |
| SP02 | 0.024 | nM | 3.69 | nM |
| SP03 | 0.225 | nM | 22 | nM |

### Example 3: The Agonist-Stabilizing Domain Fusion Proteins and the Luciferase Assay

In order to prolong the half-life of the agonist and compensate for the possible reduced activity, SP01 and SP02 were fused to the stabilizing domain.

AmG3 was fused to the GLP1 analogue SP01 or as follows:
P46 (SEQ ID NO: 29-27): SP01 was fused to the N-terminus of amG3 light chain via linker GGGGSGGSGGSGGGGS.

GLP1/GIP receptor co-agonist analogue SP02 was fused to amG2 or bispecific antibodies of GLP-1R(ahG1) and GIPR(amG2) as follows:
P28 (SEQ ID NO: 38-39): SP02 was fused to the N-terminus of amG2 light chain via a linker: GGGGSGGGGSGGGGSGGS;
P26 (SEQ ID NO: 40-43): SP02 was fused to the N-terminus of bispecific antibodies of ahG1 and amG2 light chains via the linker GGGGSGGGGSGGGGS.

The above mentioned fusion proteins were tested for their ability to stimulate cAMP production in human GLP-1R cell lines (HEK293/GLP1R/CRE-LUC), human GIPR cell lines (HEK293/hGIPR/CRE-LUC) and mouse GIPR cell lines (HEK293/mGIPR/CRE-LUC). The results of the HTRF CAMP bioassay were shown in Table 2.

TABLE 2
EC₅₀ for the agonist-stabilizing domain fusion proteins

| | Cell Lines | | | | |
|---|---|---|---|---|---|
| | hGLP-1R | | hGIPR | | mGIPR |
| | EC50 | | EC50 | | EC50 |
| P19 | 0.35 | nM | 0.19 | nM | 3.58 nM |
| P26(SEQ ID NO: 40-43) | 0.12 | nM | 48 | nM | 0.35 nM |
| P28(SEQ ID NO: 38) | 1.25 | nM | 69 | nM | 0.42 nM |

| | | | | |
|---|---|---|---|---|
| P46(SEQ ID NO: 27-29) | 0.034 | nM | — | *11.2 nM(IC₅₀) |

As shown in the Table 2, due to the amG2 is a mGIPR specific binding antibody, P26 and P28 show EC₅₀ values of 48 nM and 69 nM, respectively, for hGIP1R activation, while the EC₅₀ values increase to 0.35 nM and 0.42 nM, respectively, for mGIP1R activation, due to the tethering effects. P46 contains a mGIPR specific blocking antibody, and it shows its IC₅₀ value of 11.2 nM for mGIPR.

### Example 4: Screening of Nanobodies by Immunizing Alpacas with SP01 or SP02

GLP1 (7-37) has a very short half-life (1-2 minutes) due to its rapidly inactivation by dipeptidyl peptidase 4 (DPP4) and other proteases. It has been shown that GLP-1 and GIP receptor co-agonist can effectively reduce body weight and food intake; and these effects are significantly greater than those of the GLP1 receptor agonist. Tirzepatide, the only GLP1/GIP dual agonist drug currently available, needs to be administered weekly and has a half-life of less than one day in mice. In order to confer better resistance to protease degradation, the protecting antibodies of SP01 and SP02 were screened firstly.

Methods for preparing nanobodies that specifically bind to antigens or epitopes (heavy chain single-domain antibodies, VHHs) are described in the references, see, e.g., R. van der Linden et al., Journal of Immunological Methods, 240 (2000) 185-195; Li et al., J. Biol. Chem., 287 (2012): 13713-13721; Deffar et al., African Journal of Biotechnology, 8 (12), page 2645, Jun. 17, 2009; and WO 94/04678.

Antigens SP01 and SP02 with bovine serum albumin (BSA) or hemocyanin (KLH) conjugated to C-terminus were synthesized (QYAOBIO, China) for immunizing alpacas. Phage display technology was used to construct the phage display library. Phages expressing antigen-specific nanobodies were enriched after three rounds of bio-panning, and the following detailed experimental procedures as previously reported (U.S. 2022/0396616, Sanyou Biopharmaceutical Co., Ltd., Shanghai, China).

Ten nanobodies that bind well to SP01 (see Table 3) and 5 nanobodies that bind well to SP02 were selected (see Table 4). All nanobodies were fused to hIg1Fc and EC₅₀ was measured by ELISA.

TABLE 3
EC₅₀ for the nanobodies of SP01

| | EC₅₀ to dulaglutide |
|---|---|
| NB- hIg1Fc | (TRULICITY ® brand) (μg/mL) |
| P62-hIg1Fc (SEQ ID NO: 6) | 0.01872 |
| P64- hIg1Fc (SEQ ID NO: 7) | 0.073 |
| P65- hIg1Fc (SEQ ID NO: 8) | 0.1497 |
| P66- hIg1Fc (SEQ ID NO: 9) | 0.01926 |
| P67- hIg1Fc (SEQ ID NO: 10) | 0.03734 |
| P68- hIg1Fc (SEQ ID NO: 11) | 0.01816 |
| P70- hIg1Fc (SEQ ID NO: 12) | 0.003555 |
| P71- hIg1Fc (SEQ ID NO: 13) | 0.03602 |
| P72- hIg1Fc (SEQ ID NO: 14) | 0.01562 |
| P73- hIg1Fc (SEQ ID NO: 15) | 0.03308 |

TABLE 4
EC₅₀ for the nanobodies of SP02

| NB- hIg1Fc | EC₅₀ to P26(μg/mL) |
|---|---|
| P49- hIg1Fc (SEQ ID NO: 16) | 0.001513 |
| P55- hIg1Fc (SEQ ID NO: 17) | 0.09473 |
| P56- hIg1Fc (SEQ ID NO: 18) | 0.3465 |
| P58- hIg1Fc (SEQ ID NO: 19) | 0.06251 |
| P59- hIg1Fc (SEQ ID NO: 20) | 0.006416 |

The amino acid sequence of P67 is as described in SEQ ID NO: 21. The amino acid sequences of P62-P65, P66, P68-73 are as described in SEQ ID NO: 58-66.

The amino acid sequence of P56 is as described in SEQ ID NO: 34. The amino acid sequences of P49, P55, P58 and P59 are as described in SEQ ID NO: 67-70.

### Example 5: Pharmacokinetic Study of Fusion Proteins in C57BL Mice

The pharmacokinetic profiles in C57BL Mice (males with a body weight of approximately 25 g, Beijing Vital River Laboratory Animal Technology Co., Ltd.) were studied after subcutaneous (SC) administration of 30 nmol/kg of fusion proteins. Blood was drawn from the test subjects (6 mice per group) at various times at 6h, on Day1, 2, 4, 7, 10, and 14 after dosing. Plasma was collected from each sample, and analyzed by i): a nanobody-specific detection antibody (GenScript MonoRab™ Rabbit Anti-Camelid VHH CockTail, Cat #A02016), which will only detect the intact fusion proteins, or ii): an N-terminus specific ELISA which only detect active GLP-1 released (Novus Biologicals, Cat #NBP1-05180H).

For initial screening, all 9 SP01 nanobodies were fused to the N-terminus of SP01 via a 3×(G4S) linker without Xa factor sequences, and then further fused to the N-terminus of the hIgG4 Fc fragment via a 3×(G4S) linker. The pharmacokinetic results are shown in Table 5.

TABLE 5
Pharmacokinetics of nanobody-SP01 fusion proteins in mice

| nanobody-SP01 fusion proteins | T½(hour) |
|---|---|
| P62-3x(G4S)-SP01-3x(G4S)- hIgG4 Fc(SEQ ID NO: 44) | 63.5 |
| P64-3x(G4S)-SP01-3x(G4S)- hIgG4 Fc(SEQ ID NO: 45) | 145.6 |
| P66-3x(G4S)-SP01-3x(G4S)- hIgG4 Fc(SEQ ID NO: 46) | 28.7 |
| P67-3x(G4S)-SP01-3x(G4S)- hIgG4 Fc(SEQ ID NO: 47) | 88.6 |
| P68-3x(G4S)-SP01-3x(G4S)- hIgG4 Fc(SEQ ID NO: 48) | 26.9 |
| P70-3x(G4S)-SP01-3x(G4S)- hIgG4 Fc(SEQ ID NO: 49) | 56.5 |
| P71-3x(G4S)-SP01-3x(G4S)- hIgG4 Fc(SEQ ID NO: 50) | 81.8 |
| P72-3x(G4S)-SP01-3x(G4S)- hIgG4 Fc(SEQ ID NO: 51) | 43.4 |
| P73-3x(G4S)-SP01-3x(G4S)- hIgG4 Fc(SEQ ID NO: 52) | 40 |

In initial screening, all 5 SP02 nanobodies were fused to the N-terminus of SP02 via a 3×(G4S) linker without Xa factor sequences, and then further fused to the N-terminus of the amG2 light chain via a 3×(G4S) linker. The pharmacokinetic results are shown in Table 6.

TABLE 6
Pharmacokinetics of nanobody-SP02 fusion proteins in mice

| nanobody-SP02 fusion proteins | T½(hour) |
|---|---|
| P49-3x(G4S)-SP02-3x(G4S)-amG2(SEQ ID NO: 53-39) | 34.7 |
| P55-3x(G4S)-SP02-3x(G4S)-amG2(SEQ ID NO: 54-39) | 53.8 |
| P56-3x(G4S)-SP02-3x(G4S)-amG2(SEQ ID NO: 55-39) | 122 |
| P58-3x(G4S)-SP02-3x(G4S)-amG2(SEQ ID NO: 56-39) | 129.3 |
| P59-3x(G4S)-SP02-3x(G4S)-amG2(SEQ ID NO: 57-39) | 98.4 |

Based on the preliminary screening results of the above nanobodies, nanobodies P56 and P67 were selected for more detailed research.

### Example 6: Exemplary Fusion Proteins Containing SP01

To confer better resistance to protease degradation, the first part of the technology involves introducing a protecting antibody for GLP1 analogue at its N-terminus using a linker containing a factor Xa digestion site, the protecting antibody will specifically bind to the GLP1 analogue and protect it from proteolytic cleavage. This fusion is also completely resistant to DPP4 cleavage because the N-terminus of GLP1 is hidden. The active GLP1 analogue can be released by factor Xa digestion in blood. To further prolong the half-life of GLP1 fusion proteins, the second part of this technology involves fusing the protected GLP1 analogue to an immunoglobulin Fc fragment, or to a GIPR-blocking antibody (GIPR antagonist) to form a bispecific agonist of GLP1R and GIPR.

As an example, SP01-binding nanobody P67 (SEQ ID NO:21) was selected to fuse to GLP1 analogue SP01 (SEQ ID NO:2) to protect it from proteolytic cleavage. To further prolong its half-life, the above-mentioned nanobody-GLP1 fusion protein was fused to the immunoglobulin Fc fragment of IgG4 as follows:
P93 (SEQ ID NO: 22): Nanobody P67 (SEQ ID NO: 21) was fused to the N-terminus of GLP1 analogue SP01 via the linker with a factor Xa proteolytic cleavage sequence RGER: GGGGSGGGGSGGSGGSRGER, and then the above P67-GLP1 fusion protein was further fused to the N-terminus of the Fc fragment of human IgG4 via the linker: GGGGSGGGGSGGGGS.

P99 (SEQ ID NO: 23): Nanobody P67 (SEQ ID NO: 21) was fused to the N-terminus of GLP1 analogue SP01 via the linker with a factor Xa proteolytic cleavage sequence RKR: GGGGSGGGGGGSGGSGGSRKR, and then the above P67-GLP1 fusion protein was further fused to the N-terminus of the Fc fragment of human IgG4 via the linker: GGGGSGGGGSGGGGS.

Bispecific antibodies of GLP1 agonist and GIP antagonist have shown some good hypoglycemic and weight loss effects (Amgen patent). In order to have the dual specificity of GLP1 agonist and GIPR antagonist, the above P67-GLP1 fusion protein was further tethered to the N-terminus of the light chain of a GIP receptor blocking antibody including the heavy chain variable region shown by SEQ ID NO:24 and the light chain variable region shown by SEQ ID NO:25 (hereinafter referred to as “amG3”, MAbs. January-December 2020; 12(1): 1710047.)

P86 (SEQ ID NO: 26-27): Nanobody P67 (SEQ ID NO: 21) was fused to the N-terminus of GLP1 analogue SP01 via the linker: GGGGSGGGGSGGSGGSGGSRGER, and then the above fusion protein was further fused to the N-terminus of the amG3 light chain via the linker: GGGGSGGGGSGGGGSGGS.

P98 (SEQ ID NO: 28-27): Nanobody P67 (SEQ ID NO: 21) was fused to the N-terminus of GLP1 analogue SP01 via the linker: GGGGSGGGGGGGGSGGSRKR, and then the above fusion protein was further fused to the N-terminus of the amG3 light chain via the linker: GGGGSGGGGSGGGGSGGS.

### Example 7: Exemplary Fusion Proteins Containing SP02

To confer better resistance to protease degradation, SP02 dual agonist (SEQ ID NO:4) was fused to its binding nanobody P56 via a linker containing a factor Xa digestion site to protect it from proteolytic cleavage. To further prolong its half-life and compensate for the possible reduced activity, the above mentioned nanobody-SP02 fusion protein was further fused to an antibody for GIP receptor containing the heavy chain variable region shown by SEQ ID NO:30 and the light chain variable region shown by SEQ ID NO:31 (hereinafter referred to as “amG2”; Xiaoshan Min et al. Molecular mechanism of an antagonistic antibody against glucose-dependent insulinotropic polypeptide receptor. Mabs, 2020 January-December; 12(1):1710047); or further fused to a bispecific antibody of GIPR (amG2) and GLP1R, which included the heavy chain variable region shown by SEQ ID NO:32 and the light chain variable region shown by SEQ ID NO:32 (hereinafter referred to as “ahG1; U.S. Patent Application, Publication No. 20060275288 [Abbott Laboratories]).

P95 (SEQ ID NO: 35-36): Nanobody P56 (SEQ ID NO: 34) was fused to the N-terminus of SP02 via the linker: GGGGSGGSGGGGSGGSRGER, and then the above fusion protein was further fused to the N-terminus of the amG2 light chain via the linker: GGGGSGGGGSGGSGGS.

P100 (SEQ ID NO: 37-36): Nanobody P56 (SEQ ID NO: 34) was fused to the N-terminus of SP02 via the linker: GGGGSGGGGSGGGGSRKR, and then further fused to the N-terminus of the amG2 light chain via the linker: GGGGSGGGGSGGSGGS.

### Example 8: Pharmacokinetic Study of the Exemplary Fusion Proteins in Examples 6-7

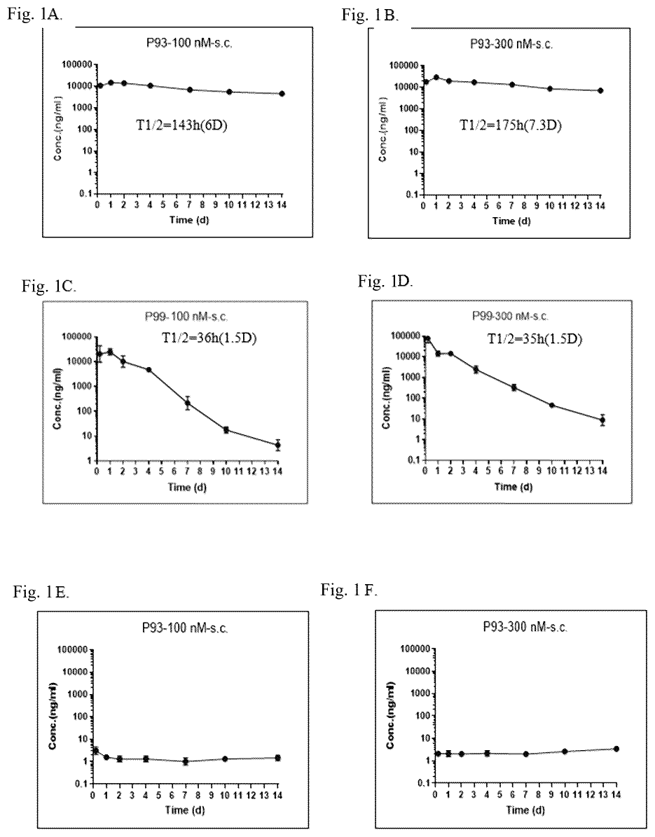

Using a similar method mentioned in example 5, the pharmacokinetic studies of the fusion proteins P93 and P99 were carried out in C57BL mice with 100 and 300 nmol/kg dosing. Both the intact fusion protein and the released active GLP1 in the collected plasma samples were detected by ELISA, and the results were shown in FIG. 1A-H. The half-life of the fusion protein P93 with a slower factor Xa digestion linker (RGER) was 143 h or 175 h at 100 or 300 nmol/kg dosing, respectively (FIG. 1A, 1B); while the fusion protein P99 with a fast factor Xa digestion linker (RKR) showed a half-life of 36 hours or 35 hours at doses of 100 or 300 nmol/kg, respectively (FIG. 1C, 1D). FIG. 1E and 1F showed that P93 can steadily release the active GLP1 fusion protein at a constant level of about 2 ng/ml; while P99 can rapidly release the active GLP1 fusion protein (FIG. 1G, 1H). Therefore, P99 has a shorter half-life compared to P93.

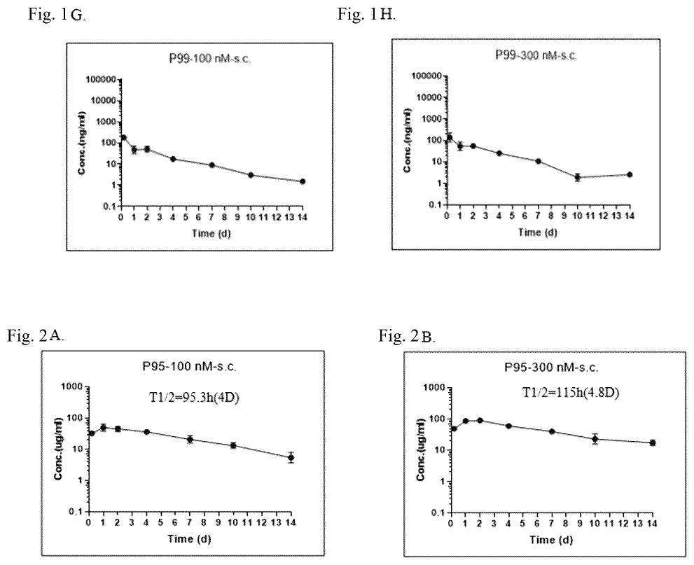

Using a similar method mentioned in Example 5, the pharmacokinetic studies of the fusion protein P95 was carried out in C57BL mice with 100 and 300 nmol/kg dosing. The concentration of the intact fusion protein P95 in the collected plasma samples was detected by ELISA, and the results were shown in FIG. 2A and FIG. 2B. The half-life of the fusion protein P95 was 95.3 hours or 115 hours at doses of 100 or 300 nmol/kg, respectively (FIG. 2A, 2B).

Notably, all protected fusion proteins showed superior pharmacokinetic properties to dulaglutide (TRULICITY® brand) and unprotected P46 in C57BL mice.

### Example 9: Effect of the Fusion Proteins on Glucose Tolerance in C57BL Mice

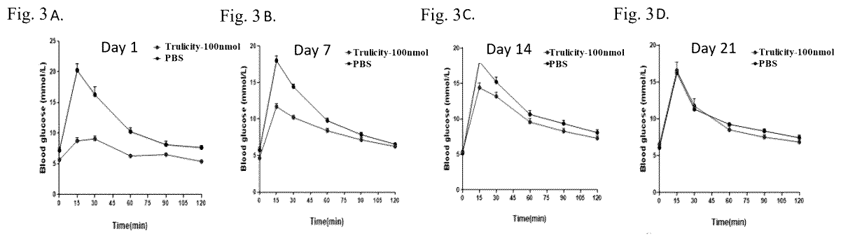

The effect of the fusion proteins on glucose tolerance was determined in C57BL mice (Beijing Vital River Laboratory Animal Technology Co., Ltd.). Each group (6 mice per group) received a single subcutaneous injection of PBS vehicle control, dulaglutide (TRULICITY® brand) (Vetter Pharma-Fertigung GmbH & Co. KG, Lilly France), P46, P28, P93, P99, P86, P98, P95, P100 at a dose of 100 or 300 nmol/kg. Intraperitoneal glucose tolerance tests (IPGTT) were performed on Day 1, 7, 14, 21, up to 28 after 6 hours fasting with tail vein blood glucose measurements at 0, 15, 30, 60, and 120 minutes. Mean±SEM of blood glucose levels at each time point were shown in FIG. 3A-I.

Single administration of the protected fusion proteins P93 and P99 at a dose of 100 or 300 nmol/kg in C57BL mice resulted in a significant decrease in glucose level for up to 28 days (FIG. 3I). P93 and P99 also showed very stable glycemic lowering effects on Day 1, 7, and 14 (FIG. 3E, 3F, 3G), while dulaglutide (TRULICITY® brand) showed very strong glucose reduction on Day 1 (FIG. 3A), but rapidly waned on Day 7 and 14 (FIG. 3B, 3C), and completely lost effect on Day 21 (FIG. 3D), which was consistent with the pharmacokinetic results that P93 and P99 can release active GLP1 in a controlled manner and maintain steady-state plasma concentrations, while dulaglutide (TRULICITY® brand) degrades rapidly.

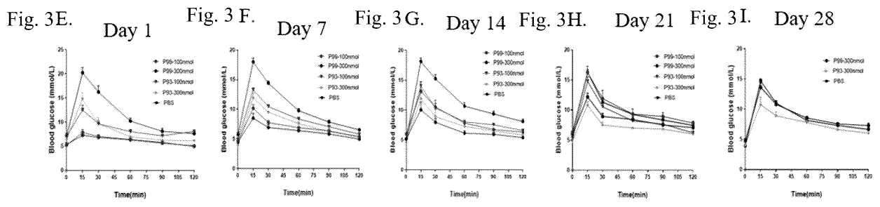

Single administration of the protected fusion proteins P86 and P98 at a dose of 100 nmol/kg showed hypoglycemic effect for a longer period of time compared to the unprotected fusion protein P46 (FIG. 4A-G). P86 and P98 improved glucose tolerance on Day 1, 7 (FIG. 4D, 4E), and up to Day 14 (FIG. 4F), while P98 still had some effect on Day 21. P46 only showed hypoglycemic effect on Day 1, 5 (FIG. 4A, 4B) and completely lost its effect on Day 10 (FIG. 4C). P86 and P98 have less glucose-reducing capacity than P93 and P99 because they contain GIP-blocking antibodies that block GIP receptor's activity.

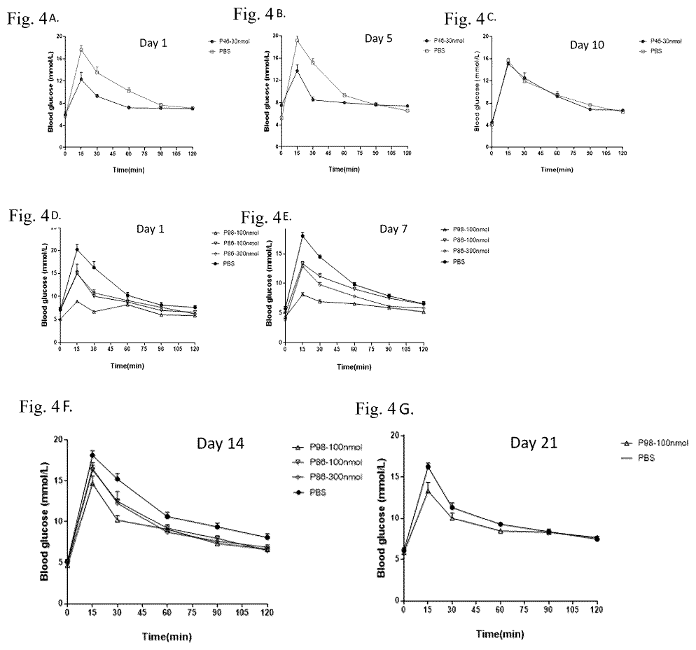

Only a slight decrease in plasma glucose was observed using the fusion protein P95 (data not shown), the possible reason is the fact that active SP02 is more difficult to be released due to the different protecting nanobody and the different N-terminus of SP02 adjacent to the factor Xa digestion site. P100 at a dose of 60 nmol/kg showed glucose-lowering effects on Day 1 (FIG. 5C) and Day 7 (FIG. 5D), while both of the unprotected fusion protein P28 and Tirzepatide P19 showed glucose-lowering effects only on Day 1 (FIG. 5A) and lost all effects on Day 7 (FIG. 5B). In our experiments, the concentration of the released active SP02 could not be measured due to lack of an N-terminus SP02-specific detection antibody.

### Example 10: Weight Loss Experiment of DIO Mice

To further characterize and understand the efficacy of the fusion proteins, body weight and metabolic parameters were measured throughout a 28-day study in DIO mice (Beijing Vital River Laboratory Animal Technology Co., Ltd.). Each group (6 mice per group) received dulaglutide (TRULICITY® brand), Tirzepatide (P19) and P46 twice weekly at a dose of 10 or 30 nmol/kg; P93, P86, P95, P98 and P99 once weekly, at a dose of 100 nmol/kg.

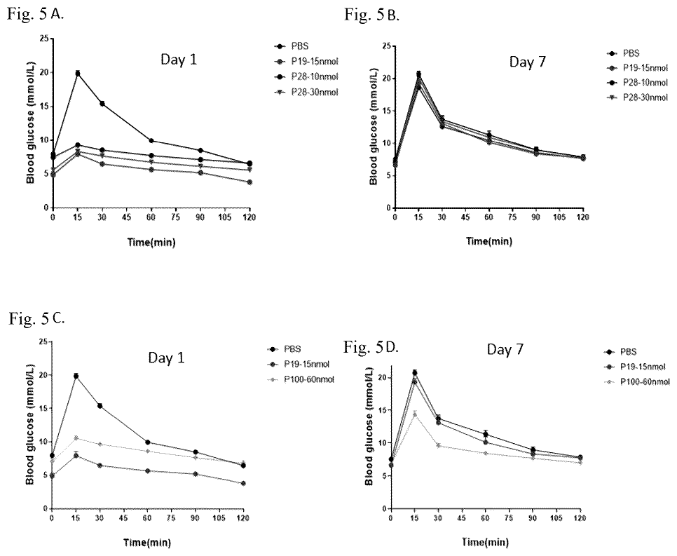

Treatment with LY3298176 (Tirzepatide, GLP1/GIP dual receptor agonist) resulted in a more significant dose-dependent decrease in body weight compared to the treatment with dulaglutide (TRULICITY® brand) (FIG. 6). Weight loss with treatment with LY3298176 was statistically significant compared to dulaglutide (TRULICITY® brand).

After 28 days, the mice treated once weekly with P93 or P99 at a dose of 100 nmol/kg showed 12.2% or 20.7% weight loss, respectively (FIG. 6). The mice treated with dulaglutide (TRULICITY®) brand) administered twice weekly with 10 or 30 nmol/kg lost 10.9% or 16.6% of their body weight (FIG. 6). The mice treated with 30 nmol/kg of Tirzepatide showed 26% weight loss. The mice treated with P86 or P98 with 100 nmol/kg showed 7.8% or 12.2% weight loss, respectively.

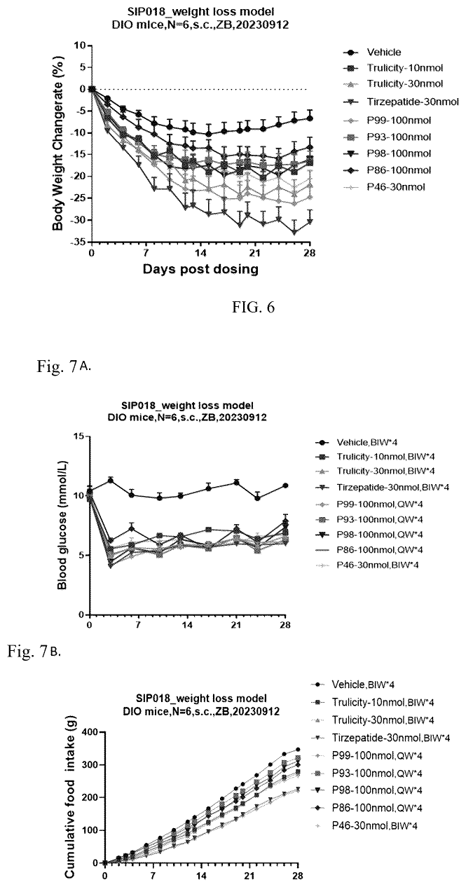

The effects of all fusion proteins on blood glucose reduction and food intake were determined in the DIO mice described above (FIG. 7A and FIG. 7B). All mice are bled after fasting for 6 hours to measure blood glucose twice weekly. Mean SEM of blood glucose levels at each time point was calculated for each group and shown in FIG. 7A. All test samples showed a significant glucose reduction, and the effects were as good as that of 30 nmol/kg dulaglutide (TRULICITY® brand) or Tirzepatide, were better than that of 10 nmol/kg of dulaglutide (TRULICITY® brand). The food intake was measured every two days (FIG. 7B), and P99 at a dose of 100 nmol/kg weekly had the greatest reduction in cumulative food intake.

It can be seen that the embodiments of the present application verified that the fusion proteins of the present application have an extended half-life and have the technical effects of weight reduction and hypoglycemia.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A glucagon-like peptide 1 (GLP1) analogue SP01, having an amino acid sequence of SEQ ID NO:2; or a GLP1/GIP receptor co-agonist analogue SP02 or SP03, having an amino acid sequence of SEQ ID NO: 4 or SEQ ID NO: 5, respectively.

2. A fusion protein comprising: a) the GLP1 analogue SP01, or the GLP1/GIP receptor co-agonist analogue SP02 or SP03 of claim 1; and b) a protecting antibody fused to an N-terminus of the GLP1 analogue or the GLP1/GIP receptor co-agonist analogue through a linker.

3. The fusion protein of claim 2, wherein the protecting antibody is a nanobody.

4. The fusion protein of claim 3, wherein the linker is 3×G4S(GGGGSGGGGSGGGGS) followed by a proteolytic cleavage sequence.

5. The fusion protein of claim 4, wherein the proteolytic cleavage sequence is Factor Xa and its variant selected from a group consisting of RGER, RKR, RGR, RSR, and RR.

6. The fusion protein of claim 3, wherein the fusion protein comprises: a) the GLP1 analogue SP01; and b) the nanobody fused to the N-terminus of the GLP1 analogue, and a sequence of the nanobody is at least 90% identity with any one of SEQ ID NOs: 21, 58, 59 and 61 to 66.

7. The fusion protein of claim 6, wherein the sequence of the nanobody is any one of SEQ ID NOs: 21, 58, 59 and 61 to 66.

8. The fusion protein of claim 3, wherein the fusion protein comprises: a) the GLP1/GIP receptor co-agonist analogue SP02; and b) the nanobody fused to the N-terminus of the GLP1/GIP analogue, a sequence of the nanobody is at least 90% identity with any one of SEQ ID NOs: 34, and 67 to 70.

9. The fusion protein of claim 8, wherein the sequence of the nanobody is any one of SEQ ID NOs: 34, and 67 to 70.

10. The fusion protein of claim 2, wherein the GLP1 analogue or the GLP1/GIP receptor co-agonist analogue is further fused to a stabilizing domain at C-terminus through a linker.

11. The fusion protein of claim 10, wherein the GLP1 analogue or the GLP1/GIP receptor co-agonist analogue is further fused to: c) an N-terminus of a Fc part of an immunoglobulin; or d) an N-terminus of a light chain or heavy chain of an antibody or an antigen-binding fragment thereof that specifically binds to a GLP1 receptor; or e) an N-terminus of a light chain or heavy chain of an antibody or an antigen-binding fragment thereof that specifically binds to a GIP receptor, or specifically blocks GIPR activity; or f) an N-terminus of a light chain or heavy chain of a bispecific antibody or an antigen-binding fragment thereof that specifically binds to both GLP1 receptor and GIP receptor, g) an N-terminus of a nanobody that specifically binds to albumin; h) an N-terminus of an albumin-binding domain (ABD) that specifically binds to albumin, at C-terminus through a linker.

12. The fusion protein of claim 11, wherein the immunoglobulin is from IgG4 or IgG2, or their variants.

13. The fusion protein of claim 10, wherein the linker is 3×G4S(GGGGSGGGGSGGGGS).

14. A nucleic acid molecule encoding the fusion protein of claim 2.

15. A recombinant expression vector capable of expressing the fusion protein of claim 2.

16. A pharmaceutical composition comprising a therapeutically effective amount of at least one agent selected from the fusion protein of claim 2, and a medicinal carrier or excipient.

17. The pharmaceutical composition of claim 16, wherein the pharmaceutical composition further comprises a second therapeutic agent.

18. A therapeutic method for preventing, improving or treating GLP1-related diseases or conditions in a subject, wherein the therapeutic method comprises administering a therapeutically effective amount of at least one agent selected from the fusion protein of claim 2.

19. The therapeutic method of claim 18, wherein the GLP1-related diseases or conditions are diabetes, obesity, or Type 2 diabetes.

20. A therapeutic method for preventing, improving or treating GLP1-related diseases or conditions in a subject, wherein the therapeutic method comprises administering a therapeutically effective amount of at least one agent selected from the pharmaceutical composition of claim 16.

21. The therapeutic method of claim 20, wherein the GLP1-related diseases or conditions are diabetes, obesity, or Type 2 diabetes.

22. A method of reducing blood sugar levels or reducing body weight in a subject, wherein the method comprises administering a therapeutically effective amount of at least one agent selected from the fusion protein of claim 2.

23. The method of claim 22, wherein the method further includes a combination with a second therapeutic agent or therapy.

24. A method of reducing blood sugar levels or reducing body weight in a subject, wherein the method comprises administering a therapeutically effective amount of at least one agent selected from the pharmaceutical composition of claim 16.

25. The method of claim 24, wherein the method further includes a combination with a second therapeutic agent or therapy.
